(19) United States
(12) Patent Application Publication
(10) Pub. No.: US 2026/0192045 A1
(43) Pub. Date: Jul. 9, 2026

(54) NEEDLE ALIGNING VEIN CLAMP DEVICE AND METHOD THEREFOR

(71) Applicant: Norma Green, Yucaipa, CA (US)

(72) Inventor: Norma Green, Yucaipa, CA (US)

(21) Appl. No.: 19/015,175

(22) Filed: Jan. 9, 2025

(51) Int. Cl. A61M 5/158

(52) U.S. Cl. CPC A61M 5/158; A61M 2005/1586; A61M 2205/0216; A61M 2210/12

(57) ABSTRACT

A needle aligning vein clamp device includes two arms and a connector. Each of the arms includes a first arm portion and a second arm portion that is resiliently flexible and pivotable with respect to the first arm portion. The connector is positioned between and couples the first arm portions together to hold the first arm portions in a fixed position with respect to the second arm portions. The connector includes a receptacle designed to releasably hold a shaft of an intravenous needle device in a position aligned with the first arm portions and with a needle of the intravenous needle device extending centrally between the arms.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TP A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (F) STATEMENT REGARDING PRIOR DISCLOSURES BY THE INVENTOR OR JOINT INVENTOR

Not Applicable

### (G) BACKGROUND OF INVENTION

### (1) Field of the Invention

The disclosure relates to vein clamps and intravenous needles and more particularly pertains to a new needle aligning vein clamp device for clamping and immobilizing a collapsed or weak vein to assist in the insertion of a needle of an intravenous needle device or catheter in the vein. The needle aligning vein clamp device allows a user to both grasp skin around a vein and at the same time align the needle for insertion in the vein.

(2) Description of Related Art including information disclosed under 37 CFR 1.97 and 1.98.

The prior art relates to vein clamps and intravenous needles that can be affixed to a patient to guide a needle. The prior art, as best understood, does not disclose a needle aligning vein clamp device includes two arms and a connector, wherein the arms have flexible ends and are coupled together by a connector, which connector includes a receptacle designed to releasably hold a shaft of an intravenous needle device with a needle of the intravenous needle device extending centrally between the arms.

### (H) BRIEF SUMMARY OF THE INVENTION

An embodiment of the disclosure meets the needs presented above in a needle aligning vein clamp device generally comprising two arms and a connector. Each of the arms includes a first arm portion and a second arm portion that is resiliently flexible and pivotable with respect to the first arm portion. The connector is positioned between and couples the first arm portions together to hold the first arm portions in a fixed position with respect to the second arm portions. The connector includes a receptacle designed to releasably hold a shaft of an intravenous needle device in a position aligned with the first arm portions and with a needle of the intravenous needle device extending centrally between the arms.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (I) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a needle aligning vein clamp device according to an embodiment of the disclosure.

FIG. 2 is a bottom view of an embodiment of the disclosure.

FIG. 3 is a rear view of an embodiment of the disclosure.

FIG. 4 is a side view of an embodiment of the disclosure in use.

FIG. 5 is a close-up view of an embodiment of the disclosure in use.

FIG. 6 is a close-up view of an embodiment of the disclosure in use.

### (J) DETAILED DESCRIPTION OF THE INVENTION

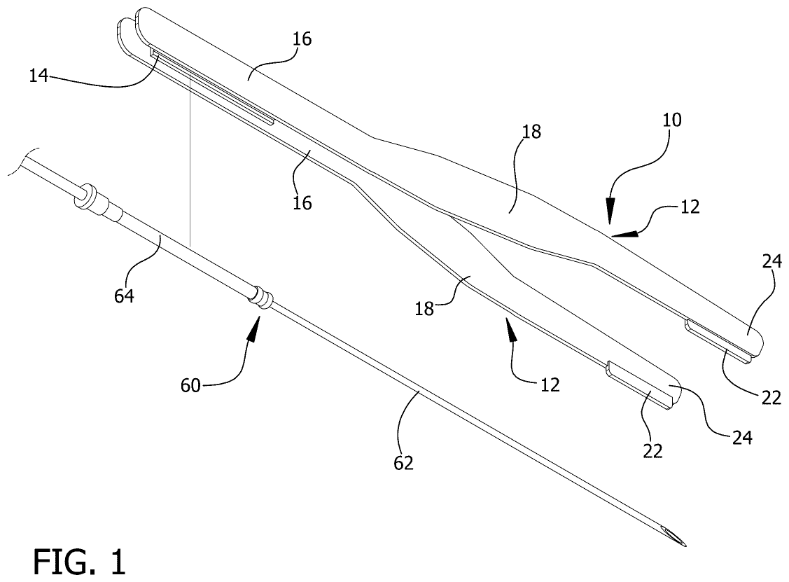

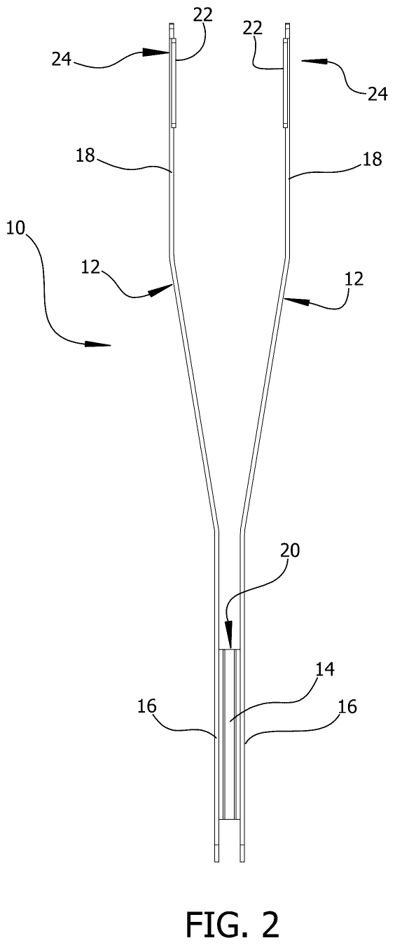

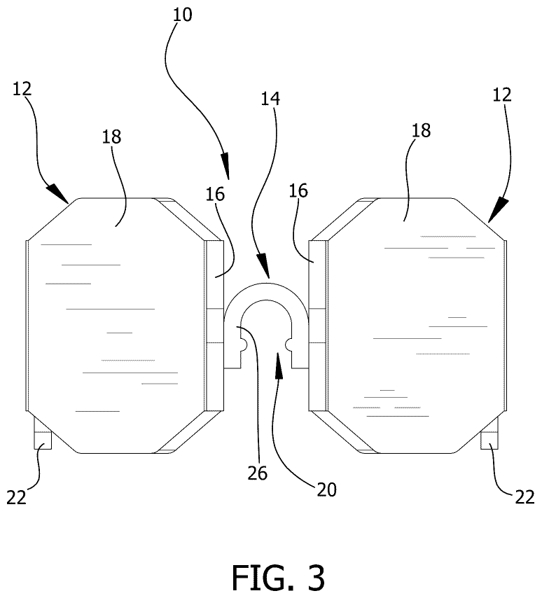

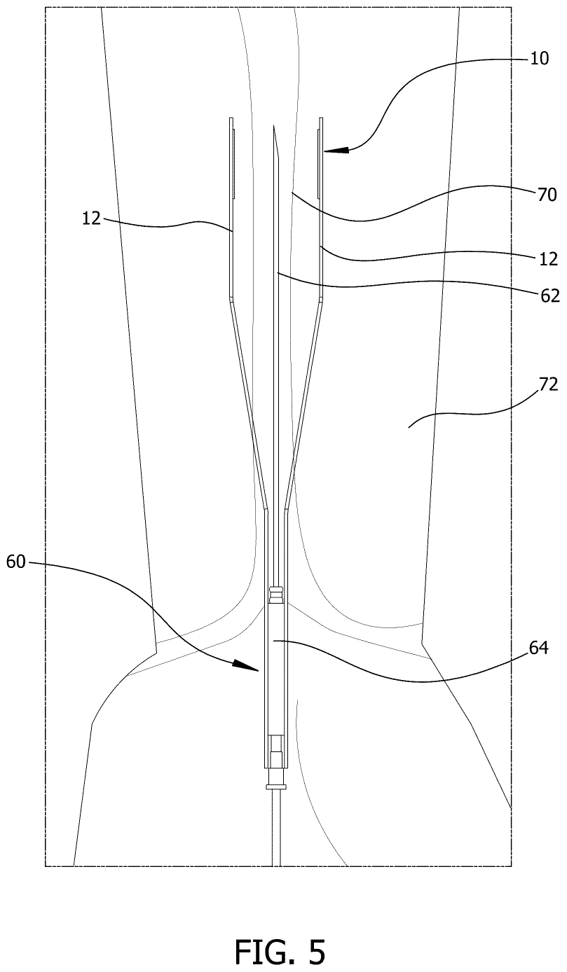

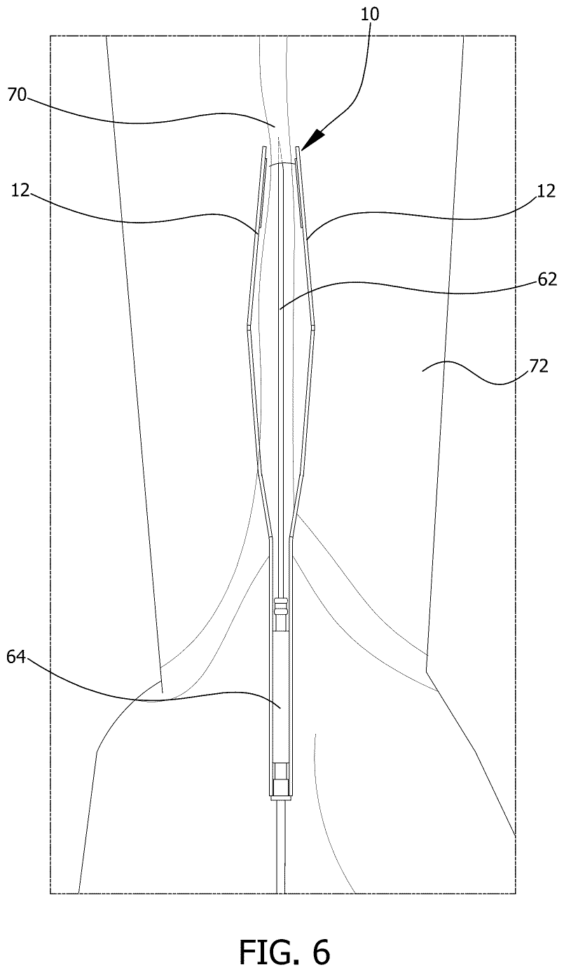

With reference now to the drawings, and in particular to FIGS. 1 through 6 thereof, a new needle aligning vein clamp device embodying the principles and concepts of an embodiment of the disclosure and generally designated by the reference numeral 10 will be described.

As best illustrated in FIGS. 1 through 6, the needle aligning vein clamp device 10 generally comprises two arms 12 and a connector 14. Each of the arms 12 includes a first arm portion 16 and a second arm portion 18 that is resiliently flexible and pivotable with respect to the first arm portion 16. The connector 14 is positioned between and couples the first arm portions 16 together to hold the first arm portions 16 in a fixed position with respect to the second arm portions 18. The connector 14 includes a receptacle 20 designed to releasably hold a shaft of an intravenous needle device in a position aligned with the first arm portions 16 and with a needle of the intravenous needle device extending centrally between the arms 12.

The arms 12 are essentially arranged in the form of tweezers or forceps. In the exemplary embodiment, each of the arms 12 is in the form of an elongated plate. Each of the second arm portions 18 is bent at an angle, though other designs, such as being curved, are within the scope of the disclosure. The needle aligning vein clamp device 10 also includes a pair of tabs 22. Each of the pair of tabs 22 is attached to a tip 24 of a corresponding one of the arms 12. Each of the pair of tabs 22 projects from its corresponding one of the pair of arms 12 to concentrate gripping forces adjacent an end of a needle of an intravenous needle device extending centrally between the arms 12.

The receptacle 20 is in the form of a C-shaped clip 26, or possibly U-shaped clip, designed to receive a tubular portion of a shaft of an intravenous needle device. The C-shaped clip 26 is resiliently flexible. The C-shaped clip 26 is in the form of an elongated tube, though the C-shaped clip 26 could be broken up into multiple C-shaped clips 26 or sections. However, in other possible embodiments, the receptacle 20 could be other shapes and sizes as would be suitable for different designs of intravenous needle device, as are well known in the medical industry. The receptacle 20 can be varied in essentially any manner provided the receptacle 20 centers the needle of the intravenous needle device in between the arms 12 with the end of the need adjacent the tips 24 of the arms 12.

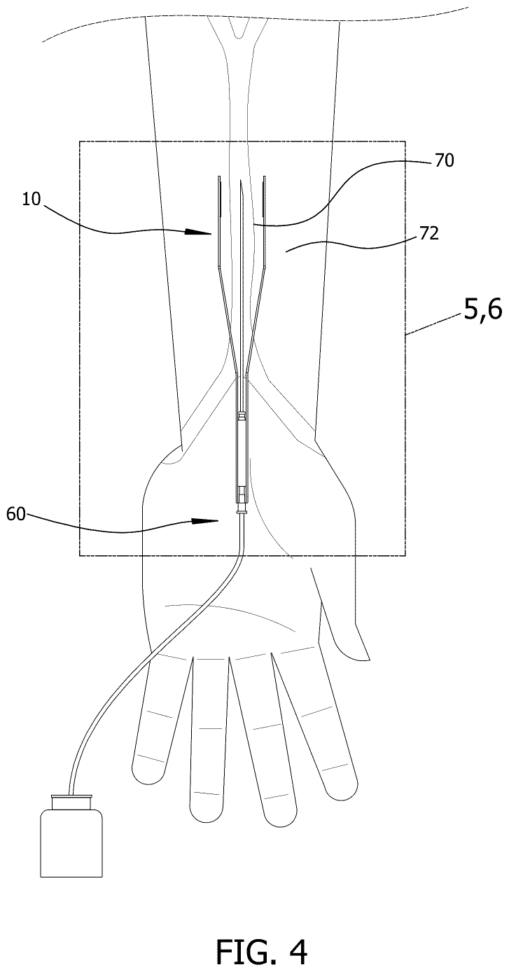

As shown in FIGS. 4, 5, and 6, the needle aligning vein clamp device 10 is used to assist in inserting a needle 62 of an intravenous needle device 60 into a vein 70, such as a vein 70 in a forearm 72 of a patient, especially if the vein 70 is weak or collapsed and thus difficult to access. The medical professional first connects a shaft 64 of the intravenous needle device 60 to the receptacle 20 with the needle 62 of the intravenous needle device 60 extending centrally between the arms 12. The medical professional then positions the second portions of the arms 12 about the vein 70. The medical professional then moves the second portions of the arms 12 together and squeezes the skin about the vein 70. The medical professional immobilizes the vein 70 between the second portions of the arms 12 and in alignment with the needle 62. The medical professional then advances the arms 12 forward and inserts the needle 62 into the vein 70. The tabs 22 can be used to press into the skin in a direction perpendicular to the arms 12 to further center the vein 70 and concentrate the gripping forces at the insertion point of the needle 62.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A needle aligning vein clamp device comprising:
two arms, each of said arms including a first arm portion and a second arm portion being resiliently flexible and pivotable with respect to said first arm portion; and
a connector disposed between and coupling said first arm portions together to hold said first arm portions in a fixed position with respect to said second arm portions, said connector including a receptacle being configured to releasably hold a shaft of an intravenous needle device in a position aligned with said first arm portions and with a needle of the intravenous needle device extending centrally between said arms.

2. The needle aligning vein clamp device of claim 1, wherein:
said receptacle includes a C-shaped clip configured to receive a tubular portion of a shaft of an intravenous needle device; and
said C-shaped clip is resiliently flexible.

3. The needle aligning vein clamp device of claim 2, wherein said C-shaped clip is in the form of an elongated tube.

4. The needle aligning vein clamp device of claim 1, wherein each of said arms is in the form of an elongated plate.

5. The needle aligning vein clamp device of claim 1, wherein each of said second arm portions is bent at an angle.

6. The needle aligning vein clamp device of claim 1, wherein:
the needle aligning vein clamp device further comprises a pair of tabs;
each of said pair of tabs is attached to a tip of a corresponding one of said arms; and
each of said pair of tabs projects from its corresponding one of said pair of arms to concentrate gripping forces adjacent an end of a needle of an intravenous needle device extending centrally between said arms.

7. A method of inserting a needle of an intravenous needle device into a vein, said method comprising the steps of:
providing a needle aligning vein clamp device comprising:
two arms, each of said arms including a first arm portion and a second arm portion being resiliently flexible and pivotable with respect to said first arm portion; and
a connector disposed between and coupling said first arm portions together to hold said first arm portions in a fixed position with respect to said second arm portions, said connector including a receptacle being configured to releasably hold a shaft of an intravenous needle device in a position aligned with said first arm portions and with a needle of the intravenous needle device extending centrally between said arms;
connecting a shaft of an intravenous needle device to said receptacle with a needle of the intravenous needle device extending centrally between said arms;
positioning said second portions of said arms about a vein;
moving said second portions of said arms together and squeezing skin about the vein;
immobilizing the vein between said second portions of said arms and in alignment with the needle; and
advancing said arms forward and inserting the needle into the vein.

8. A needle aligning vein clamp device comprising:
two arms, each of said arms including a first arm portion and a second arm portion being resiliently flexible and pivotable with respect to said first arm portion, wherein:
each of said arms is in the form of an elongated plate;
each of said second arm portions is bent at an angle;
a connector disposed between and coupling said first arm portions together to hold said first arm portions in a fixed position with respect to said second arm portions, said connector including a receptacle being configured to releasably hold a shaft of an intravenous needle device in a position aligned with said first arm portions and with a needle of the intravenous needle device extending centrally between said arms, wherein:
said receptacle includes a C-shaped clip configured to receive a tubular portion of a shaft of an intravenous needle device;
said C-shaped clip is resiliently flexible;
said C-shaped clip is in the form of an elongated tube; and
a pair of tabs, wherein:
each of said pair of tabs is attached to a tip of a corresponding one of said arms; and
each of said pair of tabs projects from its corresponding one of said pair of arms to concentrate gripping forces adjacent an end of a needle of an intravenous needle device extending centrally between said arms.
